(19) United States
(12) Patent Application Publication
(10) Pub. No.: US 2024/0408309 A1
(43) Pub. Date: Dec. 12, 2024

(54) AUTO-INJECTOR

(71) Applicant: Owen Mumford Limited, Woodstock, Oxfordshire (GB)

(72) Inventors: Toby Ken COWE, Woodstock, Oxfordshire (GB); Edward Thomas George BARRETT, Woodstock, Oxfordshire (GB)

(21) Appl. No.: 18/701,888

(22) PCT Filed: Oct. 7, 2022
(86) PCT No.: PCT/EP2022/077988

§ 371 Date: Apr. 16, 2024

(30) Foreign Application Priority Data
Oct. 18, 2021 (GB) 2114854.9

(51) Int. Cl. A61M 5/20; A61M 5/24; A61M 5/315; A61M 5/32

(52) U.S. Cl. CPC A61M 5/20; A61M 5/2422; A61M 5/31513; A61M 5/3234; A61M 2005/206

(57) ABSTRACT

According to an aspect of the present invention there is provided an auto-injector. The auto-injector includes a housing configured to receive or couple to a syringe; a plunger disposed within the housing; a drive means configured to rotate the plunger; a first screw thread arrangement configured to, during an insertion phase, cause axial movement of the plunger upon rotation of the plunger by the drive means; and a second screw thread arrangement configured to, during a delivery phase subsequent to said insertion phase, cause axial movement of the plunger upon rotation of the plunger by the drive means, wherein a thread pitch of the first screw thread arrangement is different from a thread pitch of the second screw thread arrangement.

### TECHNICAL FIELD

The present disclosure relates to an auto-injector for delivering a medicament from a syringe. In particular, the disclosure relates to an auto-injector which can provide differing insertion and delivery speeds, including when driven by a single motor.

### BACKGROUND

Injection devices, in particular auto-injectors, are commonly used to self-administer injections of medicament. Such auto-injectors are configured to receive or couple to a syringe containing the medicament in a cartridge, and then drive a plunger into the cartridge in order to deliver the medicament. Prior to this delivery, the auto-injector may facilitate the insertion of the needle into the injection site.

However, the requirements for the insertion of the needle and the delivery of the medicament are different. Namely, it is usually beneficial for the insertion of the needle to be a rapid, low-force movement. Insertion that is too slow, or that is performed with too much force, can be uncomfortable for the user, and may result in incorrect placement of the needle. On the other hand, it is usually beneficial for the delivery of medicament to be a slow, high-force movement. Delivery that is too fast may not result in adequate absorption of the medicament by the injection site. Delivery that is performed with too low a force may not overcome the viscosity of the medicament, resulting in little to no medicament delivery.

It is therefore advantageous to provide an auto-injector capable of providing a rapid, low-force insertion phase, and a slow, high-force delivery phase.

Furthermore, to improve usability, some auto-injectors are partially or predominantly electronic in nature. This may involve various functionalities of the auto-injector being controlled by an electronic controller, which may be located on the auto-injector or in a separate device. It may also involve the plunger, drive means, or similar, being driven by a motor. Some electronic auto-injectors address the aforementioned force and speed requirements by providing more than one motor, each motor configured to provide a different driving force and speed. Such electronic auto-injectors are heavy and cumbersome to use, as well as being more prone to breakage.

An improved auto-injector is required to provide differing speeds and forces during insertion and delivery phases.

### SUMMARY

In accordance with a first aspect of the disclosure, there is provided an auto-injector as defined in claim 1.

The auto-injector comprises: a housing configured to receive or couple to a syringe; a plunger disposed within the housing; and a drive means configured to rotate the plunger. The auto-injector further comprises a first screw thread arrangement and a second screw thread arrangement. The first screw thread arrangement is configured to, during an insertion phase, cause axial movement of the plunger upon rotation of the plunger by the drive means. The second screw thread arrangement is configured to, during a delivery phase subsequent to said insertion phase, cause axial movement of the plunger upon rotation of the plunger by the drive means. A thread pitch of the first screw thread arrangement is different from a thread pitch of the second screw thread arrangement.

The first screw thread arrangement may be configured to provide a first speed and a first torque to the plunger during the insertion phase. The second screw thread arrangement may be configured to provide a second speed and a second torque to the plunger during the delivery phase. The first speed may be greater than the second speed. The first torque may be lesser than the second torque. The differing speeds and torques may be provided by the differing thread pitch.

Advantageously, the presence of more than one screw thread arrangement, with the plunger able to engage with each thread arrangement during different phases, allows the speed and force of the plunger to be modulated during the entire injection stroke.

The housing may be openable to receive a syringe within the housing. Alternatively, the housing may comprise a coupling arrangement allowing a syringe to be secured to a front end of the housing. The housing may be a rear housing. The auto-injector may further comprise a front housing for housing the syringe. The front housing may be configured to couple to the front end of the rear housing.

The syringe may be a disposable syringe. The syringe may comprise: a cartridge for containing a medicament; a needle located at a front end of the syringe; a head located at the front end of the syringe; and/or a bung located at a rear end of the syringe. The head may be collapsible upon the application of axial force to the syringe.

The drive means may comprise a drive shaft that is rotationally coupled to the plunger. The drive shaft may be configured to remain coupled to the plunger during the use of the auto-injector. For example, the drive shaft may remain coupled to the plunger during the insertion phase, the delivery phase, the needle-retraction phase, and the needle-insertion phase (see below). The drive shaft may comprise one or more splines projecting radially inwards. The plunger may comprise one or more corresponding longitudinal channels. Each spline may be disposed within each channel, thereby forming a rotational coupling.

The drive means may further comprise a motor. The motor may be configured to rotate the drive shaft. The motor may be rotationally coupled to the drive shaft, for example, by way of toothed gears. The drive means may comprise only a single motor. Advantageously, the use of a single motor in an auto-injector reduces the weight and manufacturing cost of the auto-injector. It may also make the auto-injector less prone to failure, and make repairs simpler and cheaper.

The axial movement caused by the first screw thread arrangement and/or the second screw thread arrangement may be forward axial movement.

The insertion phase may begin upon or after actuation of the auto-injector. The auto-injector may comprise a trigger mechanism that is configured to cause actuation of the auto-injector. The trigger mechanism may comprise any form of trigger known in the art, for example, a button or a switch. The insertion phase may end upon or after a thread-engagement feature reaching a front end of the first thread. When the auto-injector contains or is coupled to a syringe, the insertion phase may comprise the syringe moving forwards relative to the housing, causing the needle to protrude from a front end of the auto-injector.

The delivery phase may begin upon or after the end of the insertion phase. The delivery phase may begin upon a thread-engagement feature reaching the front end of the first thread. The delivery phase may end upon a thread-engagement feature reaching a rear end of the second thread. When the auto-injector contains or is coupled to a syringe, the delivery phase may comprise the bung moving forwards relative to the syringe, causing the medicament to be delivered from the syringe, via the needle.

In some embodiments, a thread pitch of the first screw thread arrangement may be greater than a thread pitch of the second screw thread arrangement.

The thread pitch is defined as the distance between the crests of the thread. A greater thread pitch implies a greater distance between crests. A lesser thread pitch implies a lesser distance between crests. A thread with a greater pitch provides greater axial movement for a given rotation compared to a thread with a lesser pitch. A thread having a greater thread pitch provides faster axial motion. A thread having a greater thread pitch provides lower torque. A thread having a lesser thread pitch provides greater mechanical advantage.

Advantageously, by providing a greater thread pitch to the first thread, the plunger may move with faster speed and lower force (or torque) during the insertion phase, compared to during the delivery phase. This means that the plunger can provide faster motion to cause insertion of a needle of the syringe, and also provide lower force to the syringe during the insertion phase. It further means that the plunger can provide slower motion, operating with a higher force to act against the viscosity of medicament in the syringe during the delivery phase.

Alternatively, the thread pitch of the first screw thread arrangement may be lesser than the thread pitch of the second screw thread arrangement. Such an arrangement could be required if a slower or higher force needle insertion is more appropriate, or if a lower force delivery is more appropriate (for example, in the case of a low viscosity medicament).

In some embodiments, the first screw thread arrangement may comprise a first thread. The second screw thread arrangement may comprise a second thread. The first thread of the first screw thread arrangement may be defined by an inner surface of the housing. The second thread of the second screw thread arrangement may be defined by an outer surface of the plunger.

Alternatively, the first thread may be located on a component of the auto-injector that is coupled or fixed relative to the housing. Similarly, the second thread may be located on a component of the auto-injector that is coupled or fixed relative to the plunger.

In some embodiments, the auto-injector may further comprise an insertion collar. The insertion collar may be disposed coaxially around the plunger. The first screw thread arrangement may be provided between the housing and the insertion collar. The second screw thread arrangement may be provided between the insertion collar and the plunger.

The insertion collar may be rotationally and/or axially couplable to the plunger. By ‘rotationally and axially couplable’ it is meant that, when the insertion collar is coupled to the plunger, the plunger and insertion collar rotate and move axially together. That is, when coupled, there is substantially no relative rotation or axial movement between the plunger and the insertion collar.

The first screw thread arrangement may be configured to cause axial movement of the insertion collar relative to the housing upon rotation of the insertion collar relative to the housing. The second screw thread arrangement may be configured to cause axial movement of the plunger relative to the insertion collar upon rotation of the plunger relative to the insertion collar.

In some embodiments, the first screw thread arrangement may comprise one or more first thread-engagement features. The first thread-engagement features may be disposed on an outer surface of the insertion collar. The first thread-engagement features may be configured to engage with the first thread.

In other words, the first thread-engagement feature may be configured to, upon rotation of the insertion collar, travel along the first thread, thereby causing axial movement of the insertion collar. The first thread-engagement feature may be a cam, pin, or flange configured to engage with the first thread. Alternatively, the first thread-engagement feature may be a corresponding thread configured to engage with the first thread.

Optionally, the end of the insertion phase and/or the start of the delivery phase occurs when the first thread-engagement feature reaches a front end of the first thread. The second thread-engagement feature may be configured to travel along the second thread after the first thread-engagement feature reaches a front end and/or a rear end of the first thread.

In some embodiments, the second screw thread arrangement may comprise one or more second thread-engagement features. The second thread-engagement features may be disposed on an inner surface of the insertion collar. The second thread-engagement features may be configured to engage with the second thread.

In other words, the second thread-engagement feature may be configured to, upon rotation of the plunger, travel along the second thread, thereby causing axial movement of the plunger. The second thread-engagement feature may be a cam, pin, or flange configured to engage with the second thread. Alternatively, the second thread-engagement feature may be a corresponding thread configured to engage with the second thread.

Optionally, the end of the delivery phase occurs when the second thread-engagement feature reaches a rear end of the second thread.

While it has been described that the first thread may be located on the housing, the second thread may be located on the plunger, and the first and second thread-engagement features may be located on the insertion collar, it is contemplated that alternative arrangements exist. Such alternative arrangements may locate the first thread on the insertion collar, locate the second thread on the insertion collar, locate the first thread-engagement feature on the housing, and/or locate the second thread-engagement feature on the plunger. It will be understood that the features and advantages described herein are achievable irrespective of whether the threads or engagement features are located on the housing, plunger, or insertion collar.

For example, the first thread may be defined by an outer surface of the insertion collar, the second thread may be defined by an inner surface of the insertion collar, the first thread-engagement feature may be disposed on an inner surface of the housing, and the second thread-engagement feature may be disposed on an outer surface of the plunger.

In some embodiments, the one or more second thread engagement features may comprise one or more flanges. The one or more flanges may extend radially inwards of the inner surface of the insertion collar.

Alternatively, the second thread-engagement feature may comprise one or more pins or cams.

In some embodiments, the auto-injector may comprise a coupling mechanism configured to rotationally and/or axially couple the insertion collar to the plunger during the insertion phase. The first screw thread arrangement may be configured to cause axial movement of the plunger and the insertion collar upon rotation of the plunger by the drive means.

In some embodiments, the coupling mechanism may be configured to decouple the insertion collar from the plunger at the end of the insertion phase. The second screw thread arrangement may be configured to cause axial movement of the plunger through the insertion collar and the housing upon rotation of the plunger by the drive means.

The coupling mechanism may be configured to decouple the insertion collar from the plunger when the insertion collar reaches a front end and/or a rear end of the first thread.

In some embodiments, the coupling mechanism may comprise one or more cooperating features on the insertion collar. The coupling mechanism may further comprise one or more cooperating features on the plunger. The one or more cooperating features may be configured to prevent relative rotation of the plunger and the insertion collar until a relative torque between the plunger and the insertion collar exceeds a threshold.

In some embodiments, the one or more cooperating features may comprise one or more first protrusions on an outer surface of the plunger. The one or more cooperating features may further comprise one or more second protrusions provided on the insertion collar. The one or more second protrusions may be provided on one or more flexible fingers of the insertion collar. The one or more first protrusions may be configured to abut the one or more second protrusions upon rotation of the plunger by the drive means.

The one or more first protrusions may be located at a front end of the plunger. The one or more first protrusions may be located within the second thread of the plunger. The one or more second protrusions and the one or more flexible fingers may be located at a front end of the insertion collar.

The flexible fingers may define a part of an inner surface of the insertion collar. The one or more second protrusions may be located on the part of the inner surface defined by the flexible fingers. The one or more flanges of the insertion collar may be located on a part of the inner surface not defined by the flexible fingers. Advantageously, this allows the load of the plunger to be borne by a stronger part of the insertion collar (e.g. the flanges, not located on the flexible fingers). In other words, the flexible fingers do not bear the load of the plunger during the rotation of the plunger relative to the insertion collar.

The second protrusions may extend radially inwards further than the flanges. Advantageously, this allows the flanges to freely engage with the second thread, without being interrupted by the first protrusions, while still allowing the second protrusions to engage with the first protrusions.

A mechanical interaction between each first protrusion with each second protrusion may be sufficiently strong to withstand the relative torque between the insertion collar and the plunger provided by the first screw thread arrangement (e.g. during the insertion phase). The mechanical interaction may not be sufficiently strong to withstand a relative torque provided by the second screw thread arrangement (e.g. during the delivery phase). Advantageously, such an arrangement allows the plunger to be coupled to the insertion collar during the insertion phase (e.g. while the insertion collar is engaged with the first thread), but causes the plunger to decouple from the insertion collar at the end of the insertion phase (e.g. when the first thread-engagement feature reaches the front end of the first thread).

In some embodiments, the first screw thread arrangement may be configured to prevent further rotation of the insertion collar at the end of the insertion phase resulting in said relative torque exceeding said threshold upon rotation of the plunger by the drive means.

In some embodiments, the one or more flexible fingers may be configured to deflect radially outwards. The one or more flexible fingers may be configured to deflect radially outwards upon the relative torque exceeding said threshold, thereby allowing the one or more first protrusions to pass the one or more second protrusions.

In some embodiments, the drive means may comprise a motor. The auto-injector may further comprise a controller configured to control the motor to rotate the plunger in a first direction during the insertion phase and the delivery phase.

The controller may be configured to control the direction that the drive means rotates the plunger. The controller may be configured to control when and whether the drive means is activated. The controller may be configured to control the power and/or speed with which the drive means rotates the plunger. Alternatively, the controller may not be configured to control the power and/or speed with which the drive means rotates the plunger. Advantageously, the invention described herein allows the speed and/or torque of the plunger to be modulated without the need for the controller to be configured to do so. This allows for a simpler controller that is, in turn, less liable to failure and is easier to repair.

The controller may be configured to determine when the delivery phase is complete. The controller may be configured to determine that the delivery phase is complete responsive to determining that the cartridge no longer contains medicament. The controller may be configured to determine that the delivery phase is complete responsive to determining that the second thread-engagement feature has reached rear end of the second thread.

The controller may be configured to determine that the second-thread engagement feature has reached the rear end of the second thread responsive to detecting a current spike. The current spike may be received from the drive means, for example, from the motor. The current spike may be caused by the drive means attempting to rotate the plunger, but the plunger being unable to rotate further (e.g. because the plunger has reached its complete forward extension, because the plunger has pushed the bung to the end of the cartridge, and/or because the second thread-engagement feature has reached the rear end of the second thread).

In some embodiments, the controller may be configured to, when the delivery phase is complete, control the motor to rotate the plunger in a second direction opposite to the first direction during a needle-retraction phase and a subsequent plunger-retraction phase.

The controller may be configured to control the drive means to rotate the plunger in the second direction after the plunger re-engages with the first screw thread arrangement (e.g. after the plunger couples with the insertion collar). The controller may be configured to control the drive means to rotate the plunger in the second direction responsive to the controller: determining that the delivery phase is complete; determining that the cartridge no longer contains medicament; determining that the second thread-engagement feature reaches a rear end of the second thread; detecting a current spike; determining that the plunger has reached its complete forward extension; and/or determining that the plunger has pushed the bung to the end of the cartridge.

In some embodiments, the first screw thread arrangement may be configured to, during the needle-retraction phase, cause rearward axial movement of the plunger upon rotation of the plunger in the second direction by the drive means. The second screw thread arrangement may be configured to, during the plunger-retraction phase, cause rearward axial movement of the plunger upon rotation of the plunger in the second direction by the drive means.

The needle-retraction phase may begin upon or after the end of the delivery phase. The needle-retraction phase may end upon or after the first thread-engagement feature reaches a rear end of the first thread. When the auto-injector contains or is coupled to a syringe, the needle-retraction phase may comprise the syringe moving rearwards relative to the housing, causing the needle to retract from a front end of the auto-injector.

The plunger-retraction phase may begin upon or after the end of the needle-retraction phase. The plunger-retraction phase may end upon or after the second thread-engagement feature reaches a front end of the second thread. When the auto-injector contains or is coupled to a syringe, the plunger-retraction phase may comprise the plunger moving rearwards relative to the syringe, causing the removal of forward axial force from the syringe.

In some embodiments, the coupling mechanism is configured to rotationally and axially couple the insertion collar to the plunger during the needle-retraction phase. The first screw thread arrangement may be configured to cause rearward axial movement of the plunger and the insertion collar upon rotation in the second direction by the drive means.

The plunger may comprise one or more further first protrusions. The one or more further first protrusions may be located at a rear end of the plunger. The one or more further first protrusions may be located on the plunger such that, upon the second thread-engagement feature reaching the rear end of the second thread, the second protrusions on the insertion collar couple with the one or more further first protrusions. In other words, the second protrusions may ‘ride over’ the one or more further first protrusions, thereby coupling the plunger to the insertion collar.

In some embodiments, the coupling mechanism may be configured to decouple the insertion collar from the plunger at the end of the needle-retraction phase. The second screw thread arrangement may be configured to cause rearward axial movement of the plunger and the insertion collar upon rotation in the second direction by the drive means.

In some embodiments, the auto-injector may further comprise a syringe. The syringe may comprise: a cartridge for containing a medicament; a needle located at a front end of the syringe; and/or a bung located at a rear end of the syringe.

The syringe may further comprise a head at a front end of the syringe. The needle may be disposed within the head. The head may be collapsible upon the application of axial force to the syringe. The head may be configured to collapse during the insertion phase. Forward axial movement of the plunger may cause the plunger to apply axial force against the bung, causing the cartridge to move forwards relative to the head, causing the head to collapse. The head may collapse during the delivery phase, for example, at the start of the delivery phase. The collapsing of the head may cause a rear end of the needle to enter a front end of the cartridge, thereby causing fluidic contact between the needle and the medicament.

The syringe may further comprise a septum between the needle and the cartridge. The collapsing of the head may cause the rear end of the needle to pierce the septum. Advantageously, the septum provides resistance against medicament delivery, allowing initial forward axial movement of the plunger against the bung to cause forwards movement of the cartridge (rather than medicament delivery). Upon piercing of the septum by the needle, the resistance is removed, allowing medicament delivery.

In some embodiments, the auto-injector may further comprise a retraction spring configured to bias the syringe and/or the needle rearwards.

The retraction spring may be disposed around the head of the syringe. A front end of the spring may abut a front end of the front housing. A rear end of the spring may abut against a front end of the cartridge. Application of forward axial force against the syringe (e.g. by the plunger against the bung) may cause the retraction spring to compress. Removal of forward axial force from the syringe (e.g. upon retraction of the plunger) may allow the retraction spring to expand. Expansion of the retraction spring may cause expansion of the head, causing the needle to be removed from fluidic contact with the medicament. Further expansion of the retraction spring may cause the needle to retract from the front end of the auto-injector.

In some embodiments, a front end of the plunger may abut the bung such that forward movement of the plunger causes forward movement of the bung relative to the housing.

### BRIEF DESCRIPTION OF THE DRAWINGS

Exemplary embodiments will now be described with reference to the Figures, in which:

FIG. 1A is a semi-transparent side view of an auto-injector having a plunger in an initial position, the auto-injector being coupled to a syringe;

FIG. 1B is a cross-sectional side view of the auto-injector of FIG. 1A, the auto-injector not being coupled to a syringe;

FIG. 2 is a perspective view of a drive shaft for use in an auto-injector;

FIG. 3 is a perspective view of a plunger for use in an auto-injector;

FIG. 4A is a perspective view of an insertion collar for use in an auto-injector;

FIG. 4B is a front view of the insertion collar of FIG. 4A;

FIG. 4C is a front view of the insertion collar of FIG. 4A, with a plunger visible within the insertion collar;

FIG. 5A is a cross-sectional side view of an auto-injector and a syringe in an initial position;

FIG. 5B is a cross-sectional side view of the auto-injector of FIG. 5A, shown after an insertion phase;

FIG. 5C is a cross-sectional side view of the auto-injector of FIG. 5A, shown during a delivery phase;

FIG. 5D is a cross-sectional side view of the auto-injector of FIG. 5A, shown after the delivery phase;

FIG. 6A is a cross-sectional side view of the auto-injector of FIG. 5A, shown during a needle-retraction phase; and

FIG. 6B is a cross-sectional side view of the auto-injector of FIG. 5A, shown after a plunger-retraction phase.

### DETAILED DESCRIPTION

Generally disclosed herein are exemplary apparatus for auto-injectors. The term “auto-injector” is used herein to refer to a device configured to receive or couple to a syringe and provide mechanical assistance to a user during delivery of medicament from the syringe. The auto-injectors may be configured to couple to or receive, as well as operate, a disposable syringe. Alternatively or additionally, the auto-injectors may be configured to couple to or receive, as well as operate, a standard syringe (i.e. not a safety syringe) and/or a safety syringe.

In the following examples, the terms “forward”, “front”, and “proximal” refer to the end of the auto-injector, or component thereof, that faces the injection site. The injection site will typically be a patient's skin. In other words, the front end of the auto-injector is the end proximal to the injection site during use. Likewise, the terms “rearward”, “rear”, and “distal” refer to the non-injection-site end of the auto-injector, or component thereof. In other words, the term “rear” means distal from the injection site during use. Further, the terms “longitudinal” and “axial” are used to encompass a direction along or parallel to a longitudinal axis, running from the rear to the front, of the auto-injector. The term “radial” refers to the directions perpendicular to the axial direction. The term “clockwise” refers to clockwise motion as seen when viewing the auto-injector from the rear side, facing towards the front.

Features of the exemplary arrangement disclosed herein are described as being “coupled” to other features. This term encompasses any coupling that results in coupled features moving together in any direction. The term “coupled” also encompasses any one of a connection between features, an abutment of one feature against another, and an engagement of one feature with another, and such coupling may be direct or may be indirect, i.e. with a third feature therebetween.

In general terms, the disclosure is directed to improving the delivery of a medicament from an auto-injector by providing variable plunger speed and force over the course of a stroke. More specifically, the disclosure is directed to improving the delivery of a medicament from an electronic auto-injector, driven by a motor.

By providing a first screw thread arrangement and a second screw thread arrangement that are configured to cause axial movement of the plunger in different phases, different thread pitches may be used to provide differing speeds and forces to the plunger over the course of a stroke. This allows independent control of the speed and force of the plunger during, for example, an insertion phase (in which a needle is inserted into the injection site) and a delivery phase (in which medicament is delivered to the injection site via the needle).

In particular, such a configuration is advantageous in electronic auto-injectors driven by a motor, as this allows a single motor to be used in the auto-injector, while allowing more than one speed and force of the plunger. Advantageously, this reduces the weight, cost, and manufacturing complexity of electronic auto-injectors.

Furthermore, the first screw thread arrangement and second screw thread arrangement can be utilised in a reverse manner to cause retraction of the needle after medicament delivery. Advantageously, this allows safe removal of the needle from the injection site after delivery is complete without requiring any further manual input from the user, thereby reducing the risk of injury from the needle.

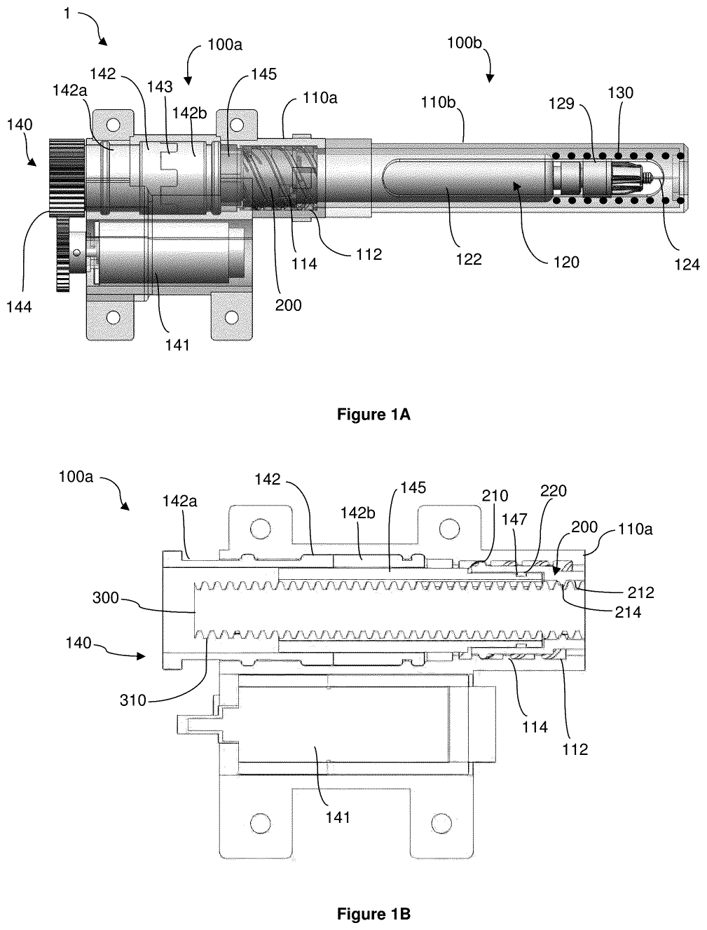

FIGS. 1A and 1B depict an exemplary auto-injector 1. The auto-injector 1 comprises a rear portion 100a having a rear housing 110a, as shown in detail in FIG. 1B. The auto-injector may further comprise a front portion 100b. The front portion 100b comprises a front housing 110b that is couplable to the rear housing 110a.

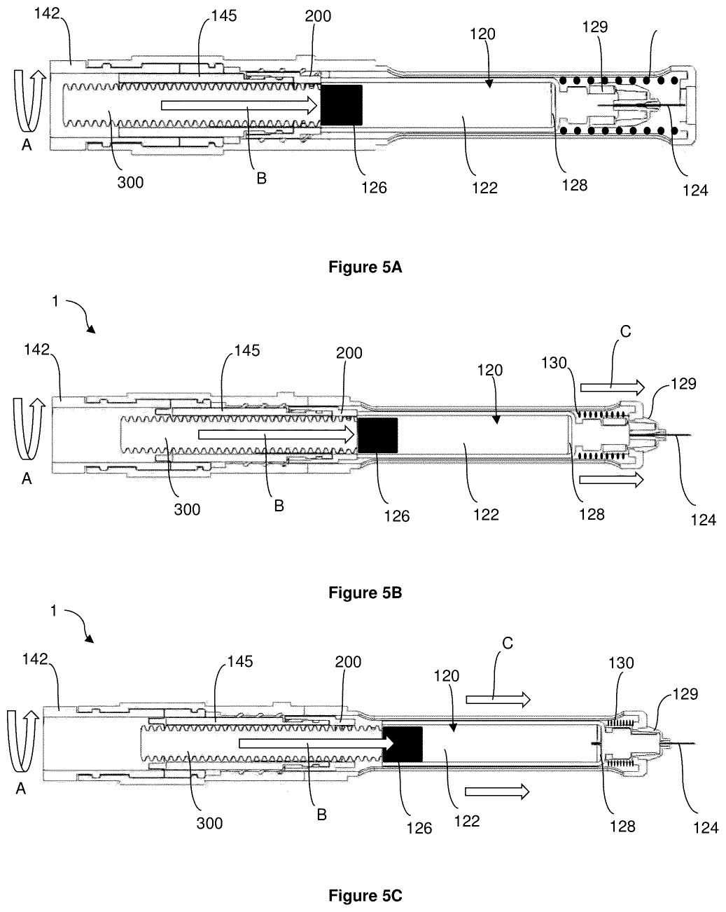

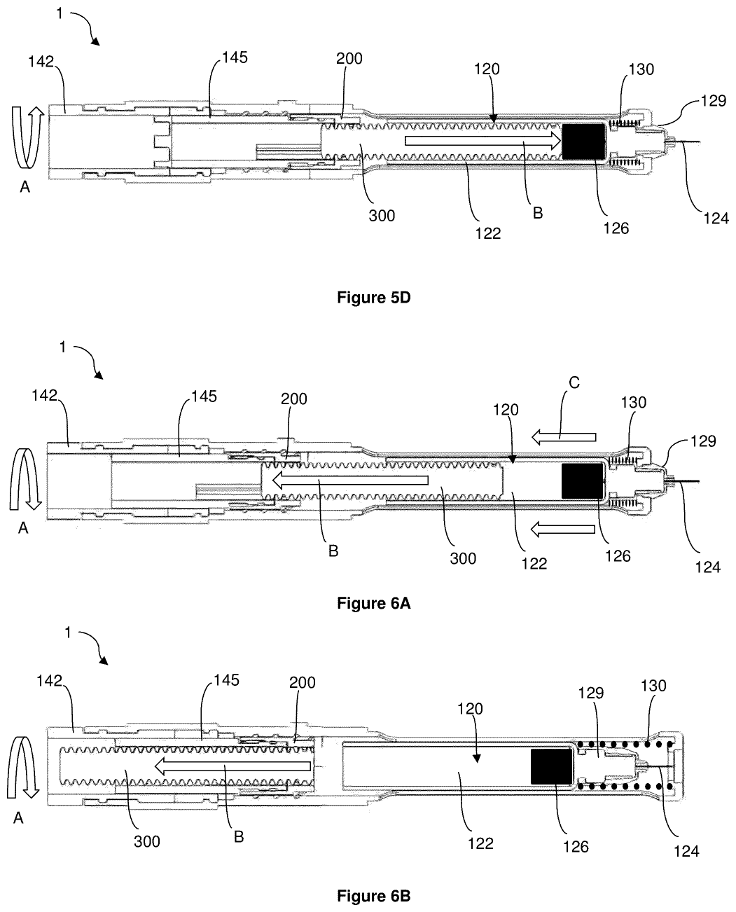

The front portion 100b further comprises a syringe 120 disposed within the front housing 110b. The syringe comprises a cartridge 122 for containing a medicament, a head 129 at a front end of the syringe, and a needle 124 disposed within the head 129, the needle 124 for delivering the medicament. The syringe 120 further comprises a bung 126 and a septum 128 (shown in FIGS. 5A-5C).

The front portion 100b further comprises a retraction spring 130 disposed within the front housing 110b at a front end of the syringe 120. The retraction spring 130 biases the syringe 120 and/or the needle 124 rearwards. In the Figures, the retraction spring 130 abuts against a front end of the front housing 110b and against a front end of the cartridge 122. Alternatively, the retraction spring 130 could be located elsewhere to rearwardly bias the needle 124 relative to the cartridge 122 (for example, the retraction spring 130 could be located within the head 129 of the syringe 120).

In some embodiments, the auto-injector 1 comprises only the rear portion 100a. In these embodiments, the front portion 100b may be supplied as a single-use, disposable component. In other words, a user may couple the front portion 100b to the rear portion 100a, actuate the auto-injector 1 to deliver medicament, and then dispose of the front portion 100b after delivery. In alternative embodiments, only the syringe 120 may be disposable, and the auto-injector 1 may comprise the rear portion 100a and the front housing 110b. In these embodiments, the user would detach the front housing 110b from the rear housing 110a, insert the syringe 120 into the front housing 110b, recouple the front housing 110b to the rear housing 110a, and deliver medicament, before disposing of the syringe 120 alone.

The rear portion 100a comprises the rear housing 110a, a drive means 140, an insertion collar 200, and a plunger 300. The rear housing 110a comprises an inner surface 112. The inner surface 112 defines a first thread 114, which will be described in greater detail below.

The drive means 140 comprises a motor 141, a drive collar 142, and a drive shaft 145. The motor 141 is operatively coupled to the drive collar 142, which is, in turn, rotationally coupled to the drive shaft 145. The drive shaft 145 is rotationally coupled to the plunger 200. The drive shaft 145 may be rotationally coupled to the plunger 200, but axially decoupled from the plunger 200. In other words, the drive shaft 145 may not rotate relative to the plunger 200, but the plunger may move axially relative to the drive shaft 145.

The motor 141 is configured to rotate the drive collar 142. The drive collar 142 is configured to rotate the drive shaft 145. The drive shaft 145 is configured to rotate the plunger 200. In the embodiment shown, the motor 141 is disposed adjacent the drive collar 142, and is coupled to the driver collar 142 by way of toothed gears 144. However, it will be appreciated that other ways of coupling the motor 141 to the driver collar 142 are possible.

The drive collar 142 comprises a rear collar 142a and a front collar 142b. The rear collar 142a is directly coupled to the toothed gears 144, and is therefore coupled to the motor 141. The front collar 142b is coupled to the rear collar 142a by castellations 143. The front collar 142b is rotationally coupled to the drive shaft 145.

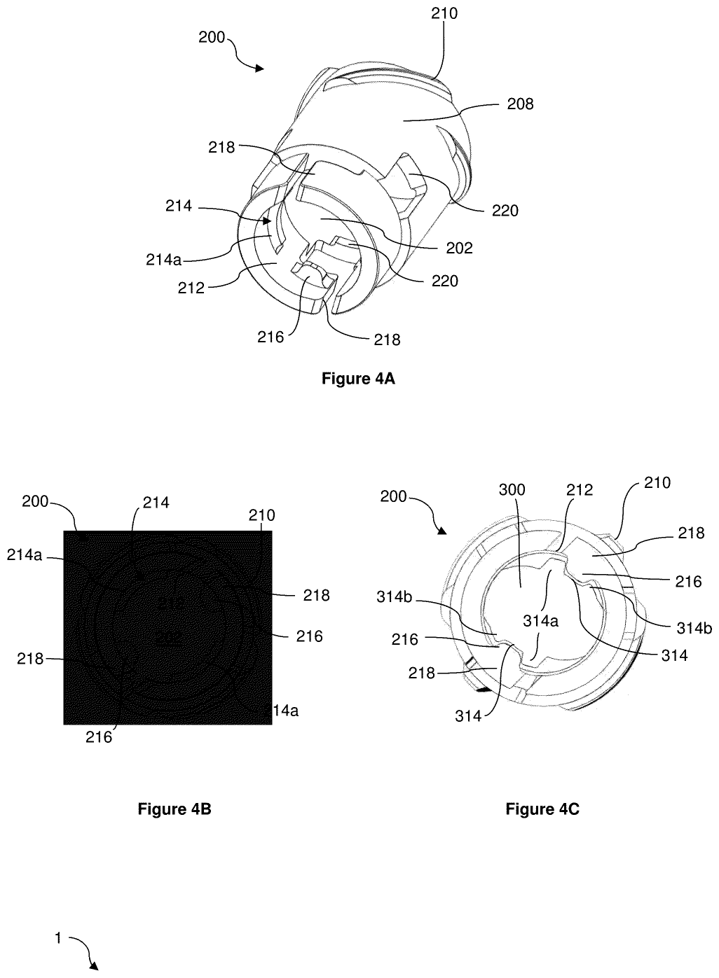

The front collar 142b may comprise one or more internal recesses (not shown) that are configured to receive one or more outer splines 148b (see FIG. 4) of the drive shaft 145. The front collar 142b may comprise one or more internal flanges (not shown) that are configured to abut an abutment 149 (see FIG. 4) on the drive shaft 145. It will be appreciated that, in alternative embodiments, the drive collar 142 may comprise only a single, integral body (rather than a rear collar 142a and a front collar 142b).

The drive collar 142 may be configured to engage with the inner surface 112 of the rear housing 110a, such that the drive collar 142 is axially fixed relative to the rear housing 110a.

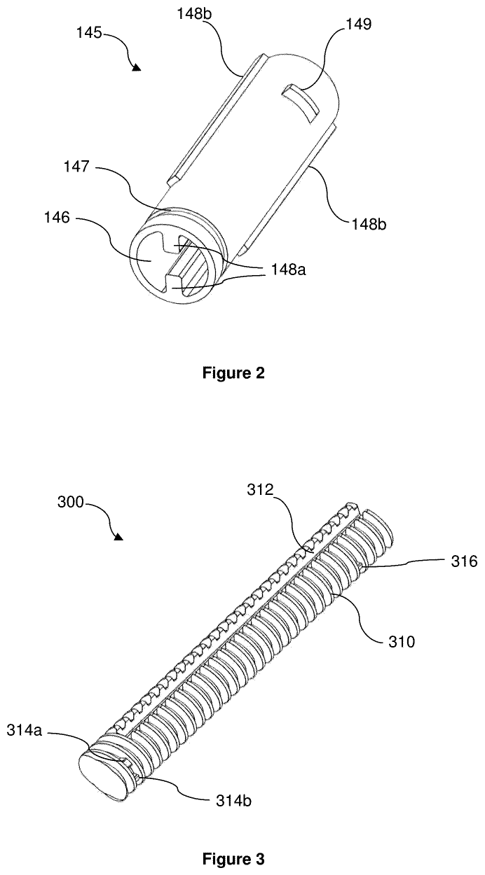

The drive shaft 145, shown in detail in FIG. 2, comprises a bore 146 for receiving the plunger 200. The drive shaft 145 further comprises a groove 147 located on an outer surface of the drive shaft 145, substantially at the front end of the drive shaft 145. When the auto-injector 1 is assembled, the groove 147 receives shoulders 220 located on the insertion collar 200 (see FIG. 4A), thereby axially coupling the drive shaft 145 to the insertion collar 200.

The drive shaft 145 further comprises one or more (in this case, two) inner splines 148a that protrude radially inwards into the bore 146. The inner splines 148a run along at least part of the length of the drive shaft 145. The inner splines 148a may run along the entire length of the drive shaft 145. The drive shaft 145 may comprise one, two, three, four, or more inner splines 148a. When the auto-injector 1 is assembled, the inner splines 148a are at least partially disposed within channels 312 (see FIG. 3) of the plunger 300, such that the plunger 300 and the drive shaft 145 are rotationally coupled.

The drive shaft 145 further comprises one or more (in this case, two) outer splines 148b that protrude radially outwards from the drive shaft 145. The outer splines 148b run along at least part of the length of the drive shaft 145. The outer splines 148b may run along the entire length of the drive shaft 145. The drive shaft 145 may comprise one, two, three, four, or more outer splines 148b. When the auto-injector 1 is assembled, the outer splines 148b are at least partially disposed within the internal recesses of the front collar 142b, such that the front collar 142b and the drive shaft 145 are rotationally coupled.

The drive shaft 145 further comprises one or more abutments 149 that protrude radially outwards from the drive shaft 145. The drive shaft may comprise one, two, three, four, or more abutments 149. When the auto-injector 1 is assembled, the abutments 149 abut the one or more internal flanges of the front collar 142b, such that the front collar 142b may not move forwards relative to the drive shaft 145 (or, equivalently, such that the drive shaft 145 may not move rearwards relative to the front collar 142b).

Turning to FIG. 3, the plunger 300 is shown in detail. The plunger 300 comprises a second thread 310 defined by an outer surface of the plunger 300. The second thread 310 will be described in greater detail below. The plunger 300 further comprises one or more (in this case, two) channels 312 that extend radially inwards from the outer surface of the plunger 300. The channels 312 run along at least part of the length of the plunger 300. The plunger 300 may comprise one, two, three, four, or more channels 312, corresponding to the number of inner splines 148a on the drive shaft 145.

Advantageously, the arrangement of inner splines 148a within the channels 312 provides a rotational coupling between the drive shaft 145 and the plunger 300, while allowing relative axial movement. Preferably, the channels 312 are open at a rear end of the plunger 300 and are closed at a front end of the plunger 300. Advantageously, the closed front end provides a stop at the front of the plunger 300 that prevents the plunger 300 from moving too far rearwards relative to the drive shaft 145. Advantageously, the open rear end allows the plunger 300 to move forwards relative to the drive shaft 145 without the inner splines 148a abutting a rear end of the channel 312.

The plunger 300 further comprises one or more (in this case, two) first protrusions 314a, 314b. The first protrusions 314a, 314b are located substantially at the front end of the plunger 300. For this reason, the first protrusions 314a, 314b may be referred to as front first protrusions 314a, 314b. The front first protrusions 314a, 314b are located within a root of the second thread 310. The plunger 300 further comprises one or more further first protrusions 316. The further first protrusions 316 are located substantially at the rear end of the plunger 300. For this reason, the further first protrusions 316 may be referred to as rear first protrusions 316. The rear first protrusions 316 are located within the root of the second thread 310. The front first protrusions 314a, 314b and the rear first protrusions 316 are configured to abut against corresponding second protrusions 216 (see FIGS. 4A-4C) on the insertion collar 200. The functionality of the first protrusions and the second protrusions will be described in detail below.

Turning to FIGS. 4A-4C, the structure of the insertion collar 200 will be described in detail. The insertion collar 200 comprises a bore 202 defined by an inner surface 212. The insertion collar 200 also comprises an outer surface 208. The insertion collar 200 comprises a first thread-engagement feature 210 disposed on the outer surface 208, and a second thread-engagement feature 214 disposed on the inner surface 212. The first thread-engagement feature 210 is configured to engage with the first thread 114 when the insertion collar 200 is disposed within the rear housing 110a. The second thread-engagement feature 214 is configured to engage with the second thread 310 when the plunger 300 is disposed within the bore 202 of the insertion collar 200.

As shown in FIGS. 4A-4C, the second thread-engagement feature may comprise one or more flanges 214a. While the insertion collar 200 is depicted as having two flanges 214a, any reasonable number of flanges may be used. The flanges 214a are configured to engage with the second thread 310 on the plunger 300.

While this configuration of the threads and the thread-engagement features is described and depicted herein, it will be understood that, equivalently, the first thread-engagement feature and the first thread are interchangeable, and the second thread-engagement feature and the second thread are interchangeable. In other words, the first thread-engagement feature 210 could be disposed on the inner surface 112 and the first thread 114 could be disposed on the outer surface 208. The second thread-engagement feature 214 could be disposed on the outer surface of the plunger 300 and the second thread 310 could be disposed on the inner surface 212.

The insertion collar 200 may further comprise shoulders 220 that project radially inward into the bore 202 of the insertion collar 200. When the auto-injector 1 is assembled, the shoulders 220 are disposed within the groove 147 on the outer surface of the drive shaft 145, thereby axially coupling the insertion collar 200 to the drive shaft 145. It will be appreciated that many other means exist for axially coupling the insertion collar 200 to the drive shaft 145.

The insertion collar 200 further comprises a coupling mechanism configured to couple to the plunger 300. The coupling mechanism may be configured to cause rotational coupling between the insertion collar 200 and the plunger 300. The functionality of the coupling mechanism will be described in greater detail below, with reference to FIG. 4C, and FIGS. 5A-6B.

The coupling mechanism may comprise one or more second protrusions 216 disposed on the inner surface 212 and protruding radially inwards of the bore 202. The coupling mechanism may further comprise one or more flexible fingers 218, each flexible finger 218 being associated with a portion of the inner surface 212. Each second protrusion 216 may be disposed on each flexible finger 218. The flexible fingers 218 are configured to deflect radially outwards upon application of a radially outwards force. Preferably, the flanges 214a are disposed on a portion of the inner surface 212 that is not associated with a flexible finger 218. Preferably, the second protrusions 216 protrude radially inwards further than the flanges 214a.

By virtue of this configuration, the second protrusions 216 may move radially outwards when the flexible fingers 218 deflect radially outwards, but the flanges 214a are not able to move radially outwards. Advantageously, this allows the second protrusions 216 to provide a coupling mechanism that can couple to and decouple from the plunger, without disengaging the second thread-engagement feature 214 (e.g. the flanges 214a) from the second thread 310.

The functionality of the coupling mechanism is apparent from FIG. 4C. FIG. 4C depicts a front cross-sectional view of the insertion collar 200, with the plunger 300 disposed within the bore 202. The second protrusions 216 on the insertion collar 200 are disposed between front first protrusions 314a and 314b on the plunger 300. It is clear from this Figure that the insertion collar 200 is rotationally coupled to the plunger 300 by virtue of this arrangement. In particular, if the plunger 300 is rotated, the front first protrusions 314a, 314b will abut against the second protrusion 216, thereby causing the insertion collar 200 to rotate with the plunger 300.

However, this functionality is provided while the insertion collar 200 is free to rotate (for example, when the first thread-engagement feature 210 is travelling along the first thread 114). If the insertion collar 200 is no longer free to rotate (for example, when the first thread-engagement feature 210 has reached a front or a rear end of the first thread 114), then the coupling mechanism is configured to decouple from the plunger 300.

Specifically, if sufficient torque is applied to the plunger 300, the second protrusions 216 will ‘ride over’ one of the first protrusions 314a, 314b, causing the flexible fingers 218 to bend radially outwards. This allows the second protrusions 216 to pass the first protrusions 314a, 314b. Further rotation of the plunger 300 will cause the second protrusions 216 to travel along the second thread 310 on the plunger 300, similarly to the flanges 214a. The plunger 300 and the insertion collar 200 are therefore no longer coupled, and can rotate with respect to one another.

As seen in FIG. 3, the plunger 300 comprises further first protrusions 316 at a rear end of the plunger 300. Once the plunger 300 has been sufficiently rotated for the insertion collar 200 to have moved towards the rear end of the plunger 300, the rear first protrusions 316 may couple to the coupling mechanism on the insertion collar 200 in a similar way. This allows the plunger 300 to recouple to the insertion collar 200. Upon recoupling, as described above, the plunger 300 and the insertion collar 200 are rotationally coupled to one another, such that rotation of sufficiently low torque (and assuming the insertion collar 200 is free to rotate) causes rotation of both components together.

The overall functionality of the auto-injector 1 will now be described with reference to FIGS. 5A-6B. For clarity, FIGS. 5A-6B contain fewer reference numerals than the preceding Figures, but it will be understood that equivalent features may be present, and that like numerals will be used where appropriate.

FIG. 5A depicts the auto-injector 1 in an initial position before the auto-injector 1 has been actuated. A syringe 120 is coupled to the front of the rear housing 110a. The bung 126 is in an initial position, prior to delivery, and the cartridge 122 is filled with medicament. Retraction spring 130 is in an expanded position. The plunger 300 is fully retracted into the rear portion 100a. In this position, the second protrusion 216 on the insertion collar 200 is disposed between first protrusion 314a and first protrusion 314b on the plunger 300. Therefore, the plunger 300 is rotationally coupled to the insertion collar 200. The first thread-engagement feature 210 on the insertion collar 200 is located at a rear end of the first thread 114 on the rear housing 110a.

As indicated by arrow A, upon actuation of the auto-injector 1 (and of the drive means 140), the drive collar 142 is rotated in a first direction. The exact nature of the first direction (i.e. clockwise or anti-clockwise) is unimportant. The first direction is simply the direction that matches the first thread 114, such that rotation in the first direction leads to forward axial motion of the insertion collar 200. Due to the aforementioned rotational couplings, rotation of the drive collar 142 leads to rotation of the drive shaft 145, in turn leading to rotation of the plunger 300. Due to the coupling mechanism, rotation of the plunger 300 leads to rotation of the insertion collar 200, causing the first thread-engagement feature 210 to travel along the first thread 114. The insertion collar 200 and the plunger 300 therefore move forwards by virtue of the first thread 114, as indicated by arrow B. This forward movement (i.e. as controlled by the first thread 114) defines the ‘insertion phase’ of the stroke.

The pitch of the first thread 114 may be configured to provide a particular speed and force of the plunger 300 and the insertion collar 200 during the insertion phase. For example, the pitch of the first thread 114 may be such that the plunger 300 and the insertion collar 200 move at a high speed, with a low force. In such a case, the torque provided by the first thread 114 is low. The pitch may be chosen such that the insertion is sufficiently fast so as to pierce the injection site without causing undue discomfort to the user. The pitch may also be chosen such that the insertion is sufficiently low force so as not to overcome the coupling mechanism or to cause delivery of medicament during the insertion phase.

During the insertion phase, the plunger 300 abuts the bung 126. Forward axial movement of the plunger 300 therefore causes application of force to the bung 126. Due to the viscosity of the medicament, and the low force nature of the insertion, movement of the bung 126 does not cause delivery of the medicament. Furthermore, as described above, the syringe 120 may comprise a septum 128 at the front end of the cartridge 122. This provides a seal that prevents the medicament from being delivered via the needle 124. The septum 128 thereby provides resistance against medicament delivery. Therefore, forward movement of the plunger 300 against the bung 126 causes forward movement of the entire syringe 120, relative to the front and rear housings 110a, 110b.

FIG. 5B depicts the auto-injector 1 at the end of the insertion phase. The first thread-engagement feature 210 has reached a front end of the first thread 114. As seen in this Figure, forward movement of the syringe 120 has caused compression of the retraction spring 130, as shown by arrows C. The head 129 of the syringe 120 has partially exited a front end of the front housing 110b. The needle 124 is therefore extending from the front end of the auto-injector 1. It will be appreciated that, if the auto-injector 1 was held against an injection site, this would cause insertion of the needle 124 into the injection site.

FIG. 5C depicts the auto-injector 1 at the start of the delivery phase (or at the end of the insertion phase). As mentioned above, the first thread-engagement feature 210 has reached the front end of the first thread 114. For this reason, the first thread-engagement feature 210 can no longer rotate further in the first direction. Therefore, continued rotation of the plunger 300 in the first direction (as shown by arrow A) causes the plunger 300 to decouple from the coupling mechanism of the insertion collar 200. In other words, front first protrusion 314a is rotated past second protrusion 216, with the flexible fingers 218 deflecting radially outward. Further rotation of the plunger 300 in the first direction therefore causes rotation of the plunger 300 relative to the insertion collar 200. The second thread-engagement feature 214 (e.g. flanges 214a) therefore travels along the second thread 310 located on the plunger 300. The plunger 300 therefore moves forwards by virtue of the second thread 310, as indicated by arrow B. This forward movement (i.e. as controlled by the second thread 310) defines the ‘delivery phase’ of the stroke.

The pitch of the second thread 310 may be configured to provide a particular speed and force of the plunger 300 during the delivery phase. For example, the pitch of the second thread 310 may be such that the plunger 300 moves at a low speed, with a high force. In such a case, the torque provided by the second thread 310 is high. The pitch may be chosen such that the insertion is sufficiently slow so as to provide gradual delivery of the medicament, appropriate for absorption by the injection site. The pitch may also be chosen such that the delivery is sufficiently high force to overcome the viscosity of the medicament, in order to cause delivery. It is noted that the force being ‘high’, and the speed being ‘low’, is defined relative to the speed and force provided during the insertion phase.

During the delivery phase, the plunger 300 abuts the bung 126. As described above, due to the presence of the septum 128 at the front end of the cartridge, no medicament is initially delivered at the start of the delivery phase. Instead, forward movement of the plunger 300 causes further forward movement of the cartridge 122, as shown by arrows C. The head 129 is unable to move further forwards due to its abutment with the front end of the front housing 110b. The head 129 may, therefore, be configured to collapse. Upon forward movement of the cartridge 122 relative to the head 129, the head 129 collapses and causes a rear end of the needle 124 to pierce the septum 128 at the front end of the cartridge 122. This places the needle 124 into fluidic contact with the medicament. The septum 128 no longer provides resistance against medicament delivery. Therefore, forward movement of the plunger 300 against the bung 126 causes forward movement of the bung 126 relative to the cartridge 122, leading to delivery of medicament from the needle 124.

FIG. 5D depicts the auto-injector 1 at the end of the delivery phase. The bung 126 has reached a front end of the cartridge 126, and therefore cannot travel forwards any further. Substantially all of the medicament has been delivered from the cartridge 126, via the needle 124. At, or slightly before, this point of delivery, the second protrusion 216 rides over (by way of deflection of the flexible fingers 218) the rear first protrusion 316. This causes the plunger 300 to recouple to the coupling mechanism of the insertion collar 200.

At this stage, the drive means 140 may continue to attempt to rotate in the first direction, as shown by arrow A. The bung 126, and therefore the plunger 300, cannot move any further forwards, and so this may cause a current spike at the drive means (for example, due to the motor 141 being unable to rotate further). A controller of the auto-injector 1 may detect this current spike and process this as an indication that the end of the delivery phase has been reached. Upon detection of the current spike, the controller may control the drive means 140 to rotate the drive collar 142 (and thus the plunger 300) in a second direction, opposite to the first direction, as shown by arrow A.

As mentioned above, the plunger 300 is recoupled to the coupling mechanism of the insertion collar 200. Therefore, rotation of the plunger 300 in the second direction causes rotation of the insertion collar 200 in the second direction, causing the first thread-engagement feature 210 to travel along the first thread 114 in the second direction. The insertion collar 200 and the plunger 300 therefore move rearwards by virtue of the first thread 114, as indicated by arrow B in FIG. 6A. This rearward movement (i.e. as controlled by the first thread 114) defines the ‘needle-retraction phase’ of the stroke.

During the needle-retraction phase, the plunger 300 is moved rearwards, away from the bung 126. There is, therefore, no longer a forward force applied to the bung 126 or the syringe 120. The retraction spring 130 is therefore no longer forced into a compressed state. The bias of the retraction spring 130 causes the retraction spring 130 to expand, moving the syringe 120 rearwards, as shown by arrows C. The needle 124 therefore move rearwards, into the front housing 110b. The needle 124 is therefore retracted from the injection site.

FIG. 6A depicts the auto-injector 1 at the end of the needle-retraction phase. The first thread-engagement feature 210 has reached a rear end of the first thread 114. For this reason, the first thread-engagement feature 210 can no longer rotate further in the second direction. Therefore, continued rotation of the plunger 300 in the second direction (as shown by arrow A) causes the plunger 300 to decouple from the coupling mechanism of the insertion collar 200 in a similar way to that described above. Further rotation of the plunger 300 in the second direction therefore causes rotation of the plunger 300 relative to the insertion collar. The second thread-engagement feature 214 (e.g. flanges 214a) therefore travels along the second thread 310 located on the plunger 300. The plunger 300 therefore moves rearwards by virtue of the second thread, as indicated by arrow B. This rearward movement (i.e. as controlled by the second thread 310) defines the ‘plunger-retraction phase’ of the stroke.

During the plunger-retraction phase, the plunger 300 returns to the initial position, (i.e. the position shown in FIG. 5A). The plunger 300 recouples to the coupling mechanism of the insertion collar 200 by virtue of the front first protrusions 314a, 314b.

FIG. 6B depicts the auto-injector 1 at the end of the plunger-retraction phase. The plunger 300 has reached the initial position, and it can be seen that the configuration is largely identical to that shown in FIG. 5A, except that the bung 126 has been moved to the front end of the cartridge 122. The retraction spring 130 has expanded again to retract the needle 124, as described above. At this stage, the front housing 110b can be removed and the syringe 120 and/or the entire front portion 100b can be discarded. The auto-injector 1 can be refitted with a fresh syringe and/or front portion and the process repeated for another delivery.

1. An auto-injector comprising:
a housing configured to receive or couple to a syringe;
a plunger disposed within the housing;
a drive means configured to rotate the plunger;
a first screw thread arrangement configured to, during an insertion phase, cause axial movement of the plunger upon rotation of the plunger by the drive means; and
a second screw thread arrangement configured to, during a delivery phase subsequent to said insertion phase, cause axial movement of the plunger upon rotation of the plunger by the drive means,
wherein a thread pitch of the first screw thread arrangement is different from a thread pitch of the second screw thread arrangement.

2. The auto-injector according to claim 1, wherein a thread pitch of the first screw thread arrangement is greater than a thread pitch of the second screw thread arrangement.

3. The auto-injector according to claim 1 or 2, wherein a first thread of the first screw thread arrangement is defined by an inner surface of the housing and a second thread of the second screw thread arrangement is defined by an outer surface of the plunger.

4. The auto-injector according to any one of the preceding claims and comprising an insertion collar disposed coaxially around the plunger, wherein the first screw thread arrangement is provided between the housing and the insertion collar, and wherein the second screw thread arrangement is provided between the insertion collar and the plunger.

5. The auto-injector according to claim 4 when dependent upon claim 3, wherein the first screw thread arrangement comprises one or more first thread-engagement features disposed on an outer surface of the insertion collar and configured to engage with the first thread.

6. The auto-injector according to claim 5 or claim 4 when dependent upon claim 3,
wherein the second screw thread arrangement comprises one or more second thread-engagement features disposed on an inner surface of the insertion collar and configured to engage with the second thread.

7. The auto-injector according to claim 6, wherein the one or more second thread engagement features comprise one or more flanges extending radially inwards of the inner surface of the insertion collar.

8. The auto-injector according to any of claims 4 to 7 and comprising a coupling mechanism configured to rotationally and axially couple the insertion collar to the plunger during the insertion phase, wherein the first screw thread arrangement is configured to cause axial movement of the plunger and the insertion collar upon rotation of the plunger by the drive means.

9. The auto-injector according to claim 8, wherein the coupling mechanism is configured to decouple the insertion collar from the plunger at the end of the insertion phase, wherein the second screw thread arrangement is configured to cause axial movement of the plunger through the insertion collar and the housing upon rotation of the plunger by the drive means.

10. The auto-injector according to claim 8 or 9, wherein the coupling mechanism comprises one or more cooperating features on the insertion collar and on the plunger, the one or more cooperating features configured to prevent relative rotation of the plunger and the insertion collar until a relative torque between the plunger and the insertion collar exceeds a threshold.

11. The auto-injector according to claim 10, wherein the one or more cooperating features comprise one or more first protrusions on an outer surface of the plunger and one or more second protrusions provided on one or more flexible fingers of the insertion collar, wherein the one or more first protrusions are configured to abut the one or more second protrusions upon rotation of the plunger by the drive means.

12. The auto-injector of claim 10 or 11, wherein said first screw thread arrangement is configured to prevent further rotation of the insertion collar at the end of the insertion phase resulting in said relative torque exceeding said threshold upon rotation of the plunger by the drive means.

13. The auto-injector of claim 11 or 12, wherein the one or more flexible fingers are configured to deflect radially outwards upon the relative torque exceeding said threshold, thereby allowing the one or more first protrusions to pass the one or more second protrusions.

14. The auto-injector according to any of the preceding claims, wherein the drive means comprises a motor, the auto-injector further comprising a controller configured to control the motor to rotate the plunger in a first direction during the insertion phase and the delivery phase.

15. The auto-injector according to claim 14, wherein the controller is configured to, when the delivery phase is complete, control the motor to rotate the plunger in a second direction opposite to the first direction during a needle-retraction phase and a subsequent plunger-retraction phase.

16. The auto-injector according to claim 15, wherein the first screw thread arrangement is configured to, during the needle-retraction phase, cause rearward axial movement of the plunger upon rotation of the plunger in the second direction by the drive means, and wherein the second screw thread arrangement is configured to, during the plunger-retraction phase, cause rearward axial movement of the plunger upon rotation of the plunger in the second direction by the drive means.

17. The auto-injector according to claim 16 when dependent upon claim 8, wherein said coupling mechanism is configured to rotationally and axially couple the insertion collar to the plunger during the needle-retraction phase, and wherein the first screw thread arrangement is configured to cause rearward axial movement of the plunger and the insertion collar upon rotation in the second direction by the drive means.

18. The auto-injector according to claim 17, wherein the coupling mechanism is configured to decouple the insertion collar from the plunger at the end of the needle-retraction phase, wherein the second screw thread arrangement is configured to cause rearward axial movement of the plunger and the insertion collar upon rotation in the second direction by the drive means.

19. The auto-injector according to any of the preceding claims, further comprising a syringe, the syringe having: a cartridge for containing a medicament; a needle located at a front end of the syringe; and a bung located at a rear end of the syringe.

20. The auto-injector according to claim 18, further comprising a retraction spring configured to bias the syringe and/or the needle rearwards.

21. The auto-injector according to claim 18 or 19, wherein a front end of the plunger abuts the bung such that forward movement of the plunger causes forward movement of the bung relative to the cartridge.
